(19) United States
(12) Patent Application Publication
(10) Pub. No.: US 2017/0188834 A1
(43) Pub. Date: Jul. 6, 2017

(54) IMAGING AND/OR PRESSURE MEASUREMENT CATHETER AND METHOD FOR USE THEREOF

(71) Applicant: The General Hospital Corporation, Boston, MA (US)

(72) Inventors: Weina LU, Peabody, MA (US); Tao WU, Cambridge, MA (US); Guillermo J. TEARNEY, Cambridge, MA (US)

(21) Appl. No.: 15/325,153

(22) PCT Filed: Jul. 10, 2015
(86) PCT No.: PCT/US15/39867

§ 371 Date: Jan. 10, 2017

Related U.S. Application Data
(60) Provisional application No. 62/022,791, filed on Jul. 10, 2014.

(51) Int. Cl. A61B 5/00; A61B 5/0215

(52) U.S. Cl. CPC A61B 5/0066; A61B 5/0215; A61B 5/02154; A61B 5/6851; A61B 5/6852

(57) ABSTRACT

Exemplary apparatus and method for obtaining information regarding at least one sample can be provided. For example, at least one optical data-obtaining first arrangement can be used which is configured to obtain data for the at least one sample based on a first light radiation provided from the sample(s). At least one pressure-sensing second arrangement can also be used which is configured to measure a pressure of at least one fluid that is provided at or near the sample(s) based on a second light radiation. In addition, e.g., a housing third arrangement can at least partially enclose the first and second arrangements.

### CROSS-REFERENCE TO RELATED APPLICATION(S)

This application is based upon and claims the benefit of priority from U.S. patent application Ser. No. 62/022,791, filed on Jul. 10, 2014, the entire disclosure of which is incorporated herein by reference.

### FIELD OF THE DISCLOSURE

The present disclosure relates generally to exemplary systems, methods and apparatus for providing imaging, including optical coherence tomography (OCT), and/or pressure measurement in, e.g., a single intravascular catheter device.

### BACKGROUND INFORMATION

Intravascular pressure measurements are important for interventional procedures on blood vessels. One example of such a medical procedure is the measurement of Fractional Flow Reserve (FFR) where a guide wire that contains a pressure-sensing element is inserted into the artery. Maximal hyperemia is induced, typically by for example administration of adenosine, and the pressure distal to the stenosis is measured and divided by the aortic pressure measured proximally. This FFR parameter can be used to determine whether or not an intravascular lesion should be treated in order to improve patient outcomes.

Even though FFR has proven to be a valuable interventional diagnostic measurement, there are many cases where FFR cannot be used alone to guide intervention. Sometimes artifacts in pressure measurements can give FFR values that are not necessarily indicative of the severity of an intravascular lesion. For many cases, the blockage is at least in part due to thrombosis that has occurred at the lesion site and in these situations FFR is not a true measure of lesion severity. For these and other reasons, it is helpful to obtain information about the structure of the artery wall to provide additional data to guide management.

One such intravascular structural imaging technique is optical coherence tomography (OCT). With intravascular OCT, a catheter is inserted into artery to determine microstructural features in the artery wall. Intravascular OCT has been shown to provide a significant amount of detail about the coronary artery, information that may be used to guide lesion management.

While both OCT and FFR guide wires are available as single devices, they typically are used separately, which can increase the duration, complexity, and cost of the procedure.

Therefore, there is a need to address at least some of the deficiencies and/or issues described herein above.

### OBJECTS AND SUMMARY OF EXEMPLARY EMBODIMENTS

Thus, apparatus and method according to exemplary embodiments of the present disclosure can be provided to facilitate imaging, including optical coherence tomography (OCT), and/or pressure measurement in, e.g., a single intravascular catheter device.

For example, conventionally, intravascular optical imaging methods, such as OCT and intravascular pressure measurements are obtained using two separate catheter devices. With the use of exemplary embodiments of the present disclosure, optical imaging and pressure measurements can be obtained using, e.g., in one exemplary variant, a single coronary catheter, which can simply the overall procedure, enhance operational efficiency, and improve patient safety, while providing a more comprehensive assessment of the vascular lesion under investigation.

Thus, it is one of the objects of the present disclosure is to provide both OCT imaging and pressure measurement in a single catheter device. In one exemplary embodiment, the pressure measurement can be obtained with an optical pressure measurement arrangement. In another exemplary embodiment, the exemplary device can include an optical fiber. Light or other electromagnetic radiation transmitted through the optical fiber can be used both for OCT imaging and pressure measurement. According to yet another exemplary embodiment, the same wavelengths of light or other electromagnetic radiation can be used for both the OCT imaging and the pressure measurement.

According to a further exemplary embodiment of the present disclosure, the exemplary device can further include a sheath that can be at least partially transparent to electromagnetic radiation, and which can be inserted into the vessel of interest. In one exemplary embodiment, OCT imaging can take place through this sheath. For example, the optical fiber that transmits the OCT electromagnetic radiation can be inside a driveshaft. The optical fiber can be distally terminated by, e.g., a lens and a beam-redirecting element. In another example, the driveshaft can be rotated within the sheath, spinning the beam around the artery wall. OCT axial scan lines (e.g., reflectivity depth profiles) can be obtained as the beam spins, comprising a cross-sectional OCT imaging of the vessel wall.

In a further exemplary embodiment of the device according to the present disclosure, the sheath can have an opening through which a pressure in the vessel can be transmitted. According to another exemplary embodiment, the sheath is closed but has a portion that is compliant and transduces pressure there through. In yet another embodiment, the sheath additionally contains a guide wire provision that allows the sheath to be guided over the guide wire. Such guide wire can be, e.g., an over-the-wire and/or a rapid exchange guide wire provision.

According to yet another exemplary embodiment of the device according to the present disclosure, an optical fiber can include a pressure sensor. For example, such exemplary optical fiber can be the same optical fiber that transceivers the OCT electromagnetic radiation. Further, the pressure sensing optical fiber and the OCT optical fiber can be different. In a further exemplary embodiment, the electromagnetic radiation from the pressure sensing fiber can illuminate a deformation arrangement, at least one portion of which can deform or move as a function of pressure within the vessel. This motion can be referenced to another portion of the exemplary arrangement that does not move and/or moves differently. The electromagnetic radiation can be transmitted from the moving portion to the fiber. In yet a further exemplary embodiment, this electromagnetic radiation can be combined with a further reference electromagnetic radiation and detected. Such exemplary interference between these electromagnetic radiations and the relative phase and/or the position can be used to determine the amount of motion of at least one portion of the deformation arrangement. The amount of motion can be processed (e.g., using a programmed computer arrangement) to compute the intravascular pressure. In still another exemplary embodiment of the present disclosure, the OCT imaging light can be transmitted through the deformation arrangement. Such exemplary deformation arrangement can be physically associated with the sheath, and/or can be a component of the imaging core that includes the optical fiber. The OCT electromagnetic light or radiation can be at least partially transmitted by the beam-redirecting element to the deformation arrangement.

In yet another exemplary embodiment of the device according to the present disclosure, the pressure sensing optical element can be or include a filter, a fiber Bragg grating, a polarization maintaining fiber, a Rayleigh scattering sensitive fiber, a photonic crystal fiber or the like. Certain wavelengths of light or electromagnetic radiation transmitted or reflected by the grating are dependent on pressure. The pressure can then be determined by measuring the spectral content of the returned light. In yet another exemplary embodiment, the filter can be based on Raman scattering and the intensity of the light can provide a measurement of the pressure. According to a still further exemplary embodiment, the optical fiber can be associated with a Fabry Perot device. The exemplary device can have a deformable portion that can move as a function of pressure. The motion can be determined by detecting electromagnetic radiation interference from the deformable portion.

According to a further exemplary embodiment of the device of the present disclosure, the outer sheath diameter can be small enough to not affect the pressure measurements inside the blood vessel. For example, the outer diameter can be less than 2.6 F, 1.5 F, etc.

Further, apparatus and method for obtaining information regarding at least one sample can be provided. For example, at least one optical data-obtaining first arrangement can be used which is configured to obtain data for the at least one sample based on a first light radiation provided from the sample(s). At least one pressure-sensing second arrangement can also be used which is configured to measure a pressure of at least one fluid that is provided at or near the sample(s) based on a second light radiation. In addition, e.g., a housing third arrangement can at least partially enclose the first and second arrangements.

For example, the second arrangement can be or include a deformable arrangement. The second light radiation can be the same as or different from the second light radiation. At least one portion of the second light radiation can be transmitted through the first arrangement. The third arrangement can include a deformable arrangement or an aperture.

According to yet another exemplary embodiment of the present disclosure, the second arrangement includes at least two portions. A first portion of the portions can be movable with respect to a second portion of the portions. A detector arrangement can be provided which is configured to receive a third light radiation reflected from the first portion and a fourth light radiation reflected from the second portion. The detector arrangement can be used to determine a position of the second portion with respect to the first portion based on an interference between the third and fourth light radiations. The position can be related to the pressure.

The third arrangement can comprise at least one channel that is structured to house a guidewire. A diameter of the third arrangement at a portion that at least partially encloses the first and second arrangements is less than 2.6 French, 1.5 French, etc. The first arrangement can include includes an interferometer, which can be a Fabry-Perot interferometer. The first arrangement can be further configured to perform a spectroscopy and/or an optical coherence tomography (OCT) procedure (e.g., including a time domain OCT, spectral-domain OCT and/or swept-source OCT). The first arrangement and/or the second arrangement can be rotatable within the third arrangement. The second arrangement can contain a fiber Bragg grating, a Rayleigh scattering fiber, and/or a photonic crystal fiber.

The first arrangement can be used to obtain data and the second arrangement can be used to measure pressure substantially simultaneously. The third arrangement can be sized to be insertable into a blood vessel. The second arrangement(s) can include a plurality of second arrangement position longitudinally along an extension of the third arrangement.

These and other objects, features and advantages of the exemplary embodiments of the present disclosure can become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure can become apparent from the following detailed description taken in conjunction with the accompanying Figures showing illustrative embodiments of the present disclosure, in which:

FIG. 1 is a perspective view of a distal end of an exemplary optical imaging and/or pressure measurement catheter according to an exemplary embodiment of the present disclosure;

FIG. 2a is a side cross-sectional view of the exemplary catheter shown in FIG. 1;

FIGS. 2b-2g are side cross-sectional views of different exemplary imaging core configurations of the measurement catheter according to various exemplary embodiments of the present disclosure;

FIG. 3a is a side cross-sectional view of a first exemplary embodiment of an imaging core of the measurement catheter;

FIG. 3b is a front cross-sectional view of the imaging core shown in FIG. 3a;

FIG. 3c is a side cross-sectional view of a second exemplary embodiment of the imaging core of the exemplary measurement catheter;

FIG. 3d is a front cross-sectional view of the imaging core shown in FIG. 3c;

FIG. 4 is a side cross-sectional view of the exemplary measurement catheter according to another exemplary embodiment of the present disclosure;

FIG. 5 is a side cross-sectional view of the exemplary measurement catheter according to still another exemplary embodiment of the present disclosure; and

FIG. 6a-6c are schematic diagrams of exemplary connections of the exemplary measurement catheter to a system console according to various exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures, or the appended claims.

### DETAILED DESCRIPTION OF EXEMPLARY EMBODIMENTS

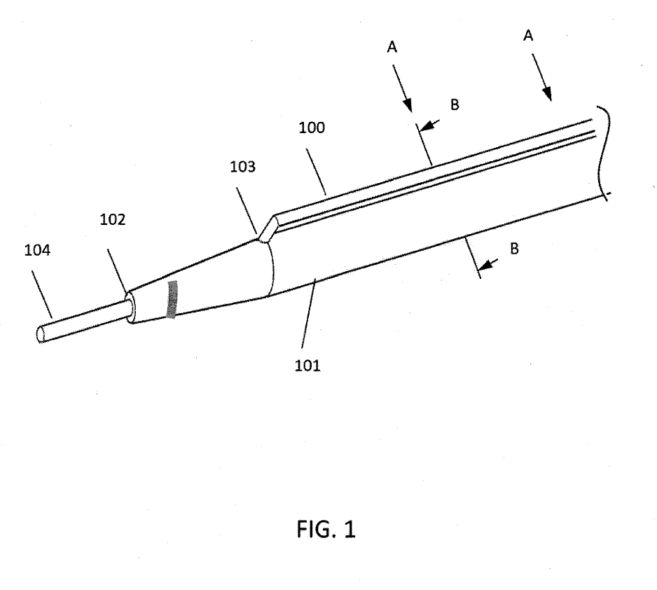

FIG. 1 shows a distal end of the exemplary optical imaging and/or pressure measurement catheter 100 according to an exemplary embodiment of the present disclosure. For example, a guide wire 104 that can be utilized, configured and/or structured for positioning an optical coherence tomography (OCT) imaging probe can be inserted through a guide wire entry port 102, and exiting of a guide wire exit port 103. OCT imaging can be facilitated, in which the light passes through an optical imaging sheath 101 and a ball lens imaging core is pulled back at a certain distance.

Various pressure measurements, including, e.g., FFR, can be obtained either simultaneously or sequentially over time. According to certain exemplary embodiments of the present disclosure, OCT procedures can operate by, e.g., interfering light or other electro-magnetic radiation reflected from a sample (e.g., including but not limited—an artery) with an additional electromagnetic radiation. The interference signal can be detected and processed (e.g., by a computer) to determine the axial reflectivity of the vessel wall. In a further exemplary embodiment of the present disclosure, the OCT signal can be collected in Time-Domain OCT (TD-OCT), where the path length delay of the reference light is changed in order to probe different distances within the artery wall. In another exemplary embodiment, the interference pattern can be detected as a function of wavelength of the electromagnetic radiation, e.g., Fourier Domain OCT. In a further exemplary embodiment of the present disclosure, which can include spectral domain OCT (SD-OCT), a broad bandwidth light source can be used as the source of electromagnetic radiation, and the interference is detected spectrally using a spectrometer.

In yet another exemplary embodiment of the present disclosure, a wavelength swept laser can be used as the source of the electromagnetic radiation, and the wavelength-dependent interference is detected as a function of time, e.g., swept-source OCT (SS-OCT) or optical frequency domain (FD) imaging (or optical frequency domain Interferometry—OFDI). For exemplary forms of FD-OCT, the reflectivity as a function of depth can be obtained, following a Fourier transformation of the spectral interference pattern. An image can be formed by scanning the beam over the sample and compiling multiple axial reflectivity profiles as a function of the beam's position. In one example, the beam can usually be focused on the sample using a lens, and redirected in a direction that is substantially perpendicular to the catheter's axis so that it illuminates the sample (e.g., the artery wall) to the side of the catheter.

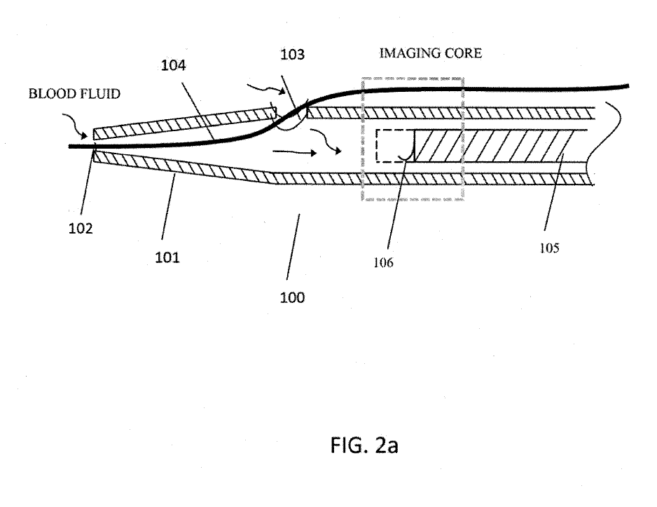

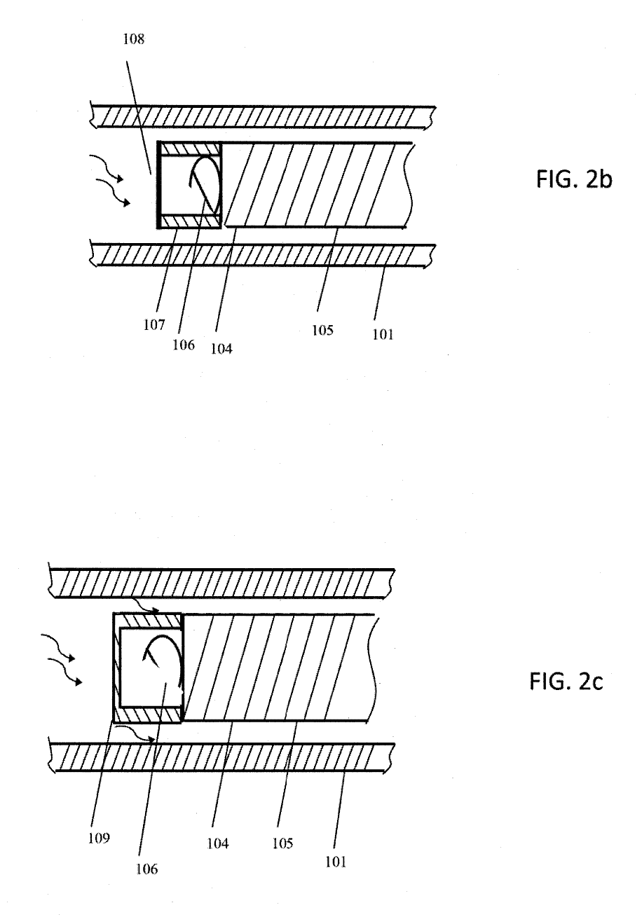

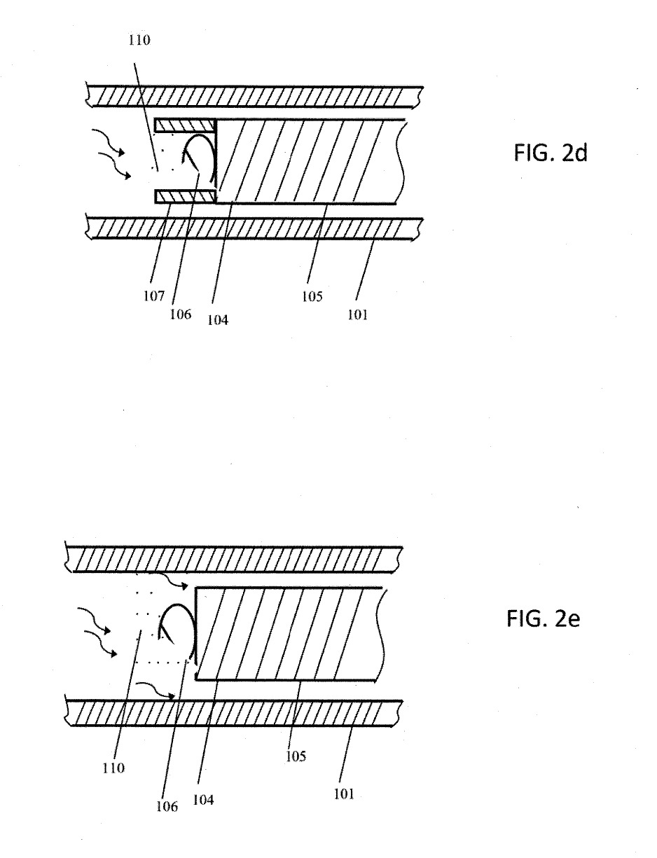

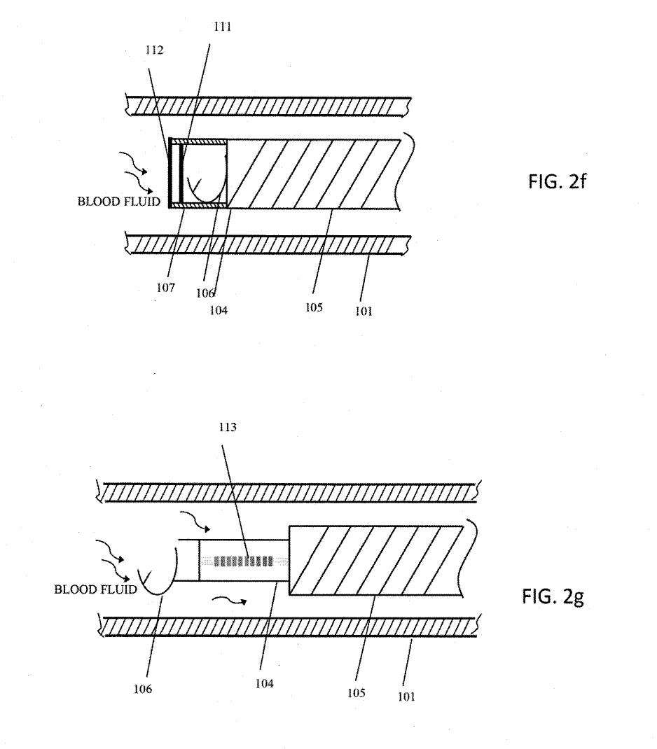

FIG. 2a illustrates an exemplary embodiment of the measurement catheter 100 with the sheath 101 that has the entry port 102 and the exit port 103 as openings so that the pressure in the vessel can be transmitted there through. For example, when the blood flows inside of the sheath 101, one or more pressure measurements can be obtained. In yet another embodiment, multiple openings can be provided in the sheath 101 and multiple pressure measurement configurations, such that the pressure can be obtained along a longitudinal extent of a vessel, e.g., including flanking the lesion under investigation.

FIGS. 2b-2g illustrate side cross-sectional views of different exemplary imaging core configurations of the measurement catheter according to various exemplary embodiments of the present disclosure. Each of these exemplary configurations of FIGS. 2b-2g illustrate an exemplary configuration that uses a ball lens 106 (e.g., a single ball lens) on a top section of the optical fiber 104 to obtain, e.g., both OCT images and pressure measurements. The fiber 104 can be threaded into a drive shaft 105, which can spin and/or pull back the imaging core to generate cross-sectional and volumetric OCT images.

FIG. 2b shows one of the exemplary configurations according to the exemplary embodiment of the present disclosure. A polished surface of the ball lens 106 can act, at least in part, as a beam splitter, which splits light or other electromagnetic radiation into at least two paths. The light or other radiation for OCT modality can be reflected by the ball lens, and passes through a rigid tubing 107 and the optical imaging sheath 101, and then impacts the coronary vessel near the side of the probe. Other light or radiation for the pressure measurement can pass through the ball lens 106 and a front soft membrane 108 that is attached to the tubing 107.

In a further exemplary embodiment of the present disclosure, the electromagnetic radiation from one surface of the membrane 108 can be combined with another electromagnetic radiation from a reference and then detected. Such interference can be analyzed (e.g., by a specially-programmed computer) with respect to an amplitude and/or a phase of the resultant radiation to determine a displacement of the compliant tubing which is then related to a pressure based on at least one of a knowledge of the mechanical properties of the tubing or a predetermined calibration function. According to yet another exemplary embodiment, a common path interferometer can be provided and/or utilized, where a first electromagnetic radiation provided from the membrane 108 is combined with a second radiation from another source where the first and second radiations follow a substantially common path. A change in the pressure in the lumen of the vessel can cause a deformation of the soft membrane 108 because the pressure can be transmitted from the vessel to inside of the catheter device through the guide wire entry port 102 and the guide wire exit port 103.

In a further exemplary embodiment of the present disclosure, the path length between the lens and the membrane 108, and the lens and the artery wall can be different, such that the image of the coronary vessel and the shape of the soft membrane 108 can be displayed in, e.g., the same image window. According to still another embodiment of the present disclosure, the signal provided from and/or associated with the membrane 108 can be used to determine the distance of the motion of the membrane 108, which can be a function of the pressure. In yet another exemplary embodiment, the motion of the membrane 108 can be determined by measuring the phase of the interference signal that is created from the combination of the reflectance from the membrane 108 and the reference arm.

FIG. 2c shows another exemplary configuration according to the exemplary embodiment of the present disclosure. In contrast to the exemplary configuration illustrated in FIG. 2b, the ball lens 106 of FIG. 2c is incorporated into a single piece of soft or compliant tubing 109. The polished surface of the ball lens 106 can act to split light into at least two paths. The light (or other electromagnetic radiation) for the OCT modality can be reflected by the ball lens 106, passed through the side of the soft tubing 109 and the optical imaging sheath 101, and then impact on the coronary vessel at the side of the probe. The other light (or electromagnetic radiation) that is used for the pressure measurement can pass through the ball lens 106 and the front section of the soft tubing 109. A change in the pressure inside the vessel can cause a deformation of the front section of the soft tubing 109. The image of the coronary vessel and the shape, motion and/or displacement of the front section of the soft tubing 109 can be displayed and/or provided in the same image window, the same data section and/or portion so that the tissue morphology and/or the pressure can be measured at the same time. According to an exemplary embodiment of the present disclosure, the electromagnetic radiation from one surface of the compliant or soft tubing 109 can be combined with another electromagnetic radiation from a reference, and then detected and interfered. This interference can be analyzed (e.g., by a specially-programmed computer) with respect to an amplitude and/or a phase of the resultant radiation to determine a displacement of the compliant tubing, which can then related to a pressure based on at least one of a knowledge of the mechanical properties of the tubing or a predetermined calibration function.

According to a further exemplary configuration at least very similar to the configuration shown in FIG. 2c, the polished surface of the ball lens 106 can be or include a reflector, which reflects light or other electromagnetic radiation unto a side path. For example, the light or other electromagnetic radiation for the OCT modality can pass through the side section of the soft tubing 109 and the optical imaging sheath 101, and then impact the coronary vessel at the side section of the exemplary probe. The reflected light or other electromagnetic radiation can also be used for the pressure measurement, since the side section of the soft tubing 109 can also be deformed by the pressure inside the vessel. There is a difference between the path length of the OCT imaging modality and the pressure measurement modality such that the shape of the coronary vessel and the shape of the front of soft tubing 109 can be detected from the same signal, and processed/displayed simultaneously, e.g., to achieve the measurement of tissue morphology and pressure at the same time. The electromagnetic radiation from one surface of the soft tubing 109 can be combined with another electromagnetic radiation from the reference and then detected. Such exemplary interference can be analyzed (e.g., using a specifically-programmed computer) with respect to an amplitude or a phase of the resultant radiation to determine a displacement of the soft tubing 109 which can then be related to a pressure based on a knowledge of the mechanical properties of the tubing and/or a predetermined calibration function.

FIG. 2d shows still another exemplary configuration according to the exemplary embodiment of the present disclosure. In this exemplary configuration, the polished surface of the ball lens 106 splits the light or another electromagnetic radiation into at least two paths. The light another electromagnetic radiation for the OCT modality can be reflected by the ball lens 106, pass through the rigid tubing 107 and the optical imaging sheath 101, and then impact the coronary vessel at the side section of the probe. Other light or further electromagnetic radiation used for the pressure measurement can be transmitted through the ball lens 106 and a front compliant material 110. A change in the pressure inside the vessel can cause deformation of the compliant material 110. There is a difference between the path length of the OCT imaging modality and the pressure measurement modality such that the shape of the coronary vessel and the shape of the compliant material 110 can be displayed in the same image window, e.g., to achieve the measurement of tissue morphology and the pressure at the same time.

FIG. 2e shows a further exemplary configuration according to the exemplary embodiment of the present disclosure. The polished surface of the ball lens 106 can split light or another electromagnetic radiation into at least two paths. The light or another electromagnetic radiation for the OCT modality can be reflected by the ball lens 106, pass through the side section of the compliant material 110 and the optical imaging sheath 101, and then impact the coronary vessel at the side section of the probe. The other light or further electromagnetic radiation for the pressure measurement can pass through the ball lens 106 and a front section of the compliant material 110. Bloods pressure changes can cause a deformation of the compliant material 110. The electromagnetic radiation from one surface of the compliant material 110 can be combined with another electromagnetic radiation from the reference and then detected. Such exemplary interference can be analyzed (e.g., with a specifically-programmed computer) with respect to the amplitude or the phase of the resultant radiation to determine a displacement of the compliant material 110, which is then related to a pressure based on a knowledge of the mechanical properties of the tubing and/or a predetermined calibration function.

According to a further exemplary configuration at least very similar to that shown in FIG. 2e, the polished surface of the ball lens 106 can redirect the light or another electromagnetic radiation into a side path. The light or the other electromagnetic radiation for the OCT modality can passes through the side section of a gel material 110 and the optical imaging sheath 101, and then impact the coronary vessel at the side section of the probe. The reflected light or another electromagnetic radiation can also be used for the pressure measurement, since the side section of the gel material 110 can also be deformed and/or be altered in shape by changes in the pressure, and can provide the pressure measurement modality. There is a difference between the path length of the OCT imaging modality and the pressure measurement modality such that the shape of the coronary vessel and the shape of the front section of the gel material 110 can be displayed in the same image window, e.g., to achieve the measurement of the tissue morphology and the pressure, at the same time. Electromagnetic radiation from one surface of the gel material 110 can be combined with another electromagnetic radiation from the reference and then detected. Such exemplary interference can be analyzed (e.g., using a specifically-programmed computer) with respect to the amplitude or the phase of the resultant radiation to determine a displacement of the gel material 110 which can then be related to a pressure based on a knowledge of the mechanical properties of the tubing and/or a predetermined calibration function.

FIG. 2f shows a still further exemplary configuration according to the exemplary embodiment of the present disclosure. For example, after the polished surface of the ball lens 106, partial light (or another electromagnetic radiation) is redirected into side path. The light (or another electromagnetic radiation) for OCT modality can pass through the side of the compliant (or gel) material 110 and the optical imaging sheath 101, and then impact the coronary vessel at the side of the probe. For obtaining a pressure measurement, e.g., the Fabry-Perot interferometer can be is utilized. After the polished surface, some light (or another electromagnetic radiation) can pass through the ball lens 106, impact the mirror 111 and reflected back to the fiber; while some light (or another electromagnetic radiation) can passes through the mirror 111, impact the deformed diaphragm 112, and then be reflected back to the fiber. The interference signal generated by the two reflected lights (or another electromagnetic radiations) can generate the signal which can be the pressure information and/or related thereto. In one exemplary case, the interferometer can be or include a common path interferometer, where the electromagnetic radiation can be reflected on the membrane or diaphragm 112, and the other from a less movable portion of the interferometer element that can act as a reference. The interference from the signals coming of these exemplary surfaces can be used to determine the deformation of the membrane, and may be related to the pressure by knowledge of the mechanical properties of the membrane or by the use of a predetermined calibration function.

FIG. 2g shows yet another exemplary configuration according to the exemplary embodiment of the present disclosure. As shown in FIG. 2g, a BRAGG grating 113 can be provided in the same fiber as the ball lens fiber. Therefore, e.g., utilizing the same fiber, the OCT imaging modality and pressure measurement modality can be achieved. According to still another embodiment of the present disclosure, a polarization maintaining fiber and/or a photonic crystal fiber can be provided in the same fiber as the ball lens fiber. In a further exemplary embodiment, the OCT electromagnetic radiation can be transceived through the same fiber.

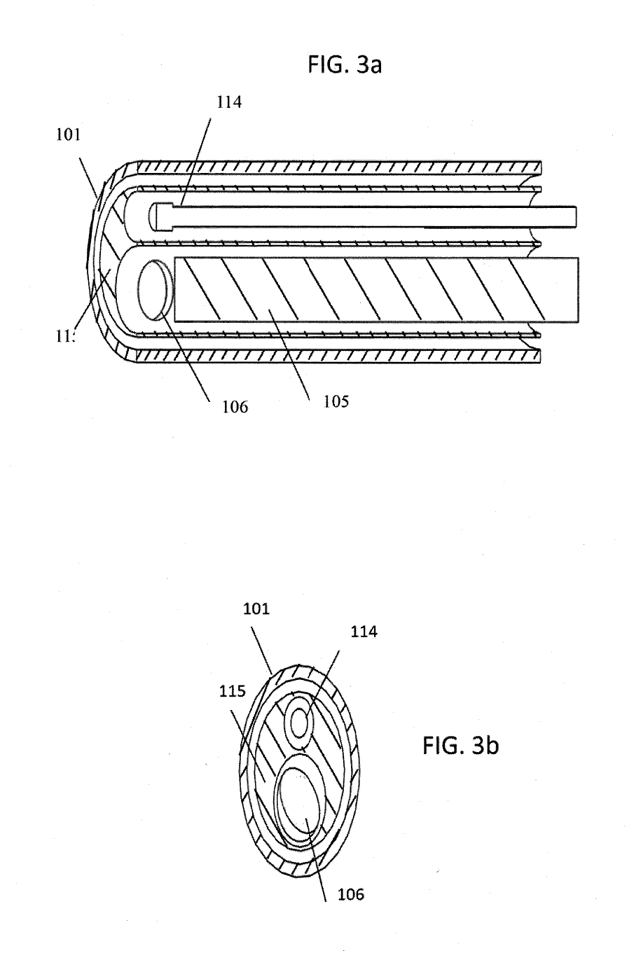

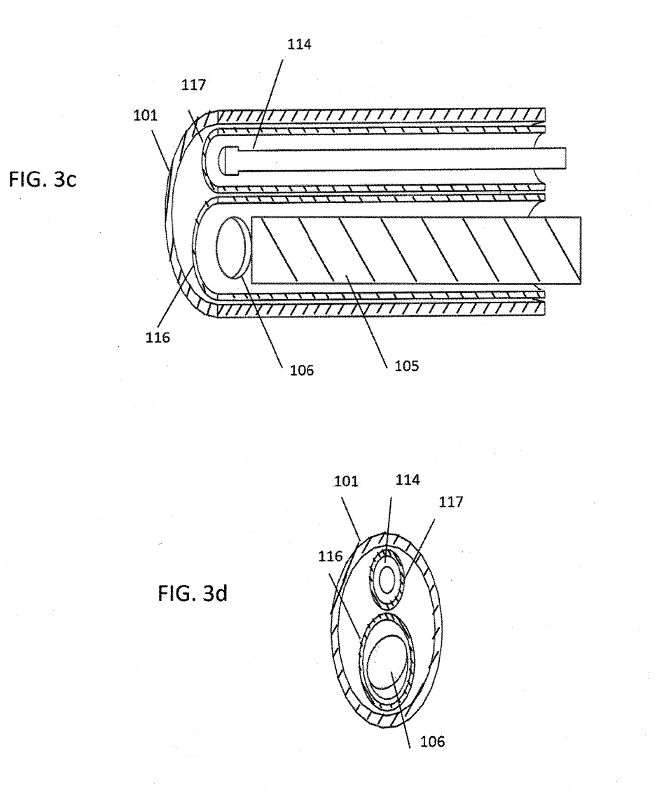

FIGS. 3a-3d illustrate additional exemplary embodiments of the present disclosure that utilize a plurality of (e.g., 2) separate optical cores. For example, OCT images can be obtained in this exemplary embodiment using a lensed imaging core 106, and the pressure measurement can be obtained by a core of a fiber-optic sensor 114. The fiber-optic sensor 114 can be or include any type of a sensor arrangement for pressure sensing, such as, e.g., a photo-elastic based sensor, a optomechanical based sensor, a fiber BRAGG grating sensor, a Fabry-Perot sensor, a polarization maintaining fiber, a photonic crystal fiber, etc. As shown in the exemplary embodiments of FIG. 3a, the optical cores can be separated in a dual lumen tubing 115. As illustrated in the exemplary embodiments of FIG. 3(c), the optical cores can be threaded into separate tubings, e.g., a tubing for an OCT imaging core 116, and a tubing for an FFR core 117. In such exemplary embodiments, a different light source or the same light source can be used.

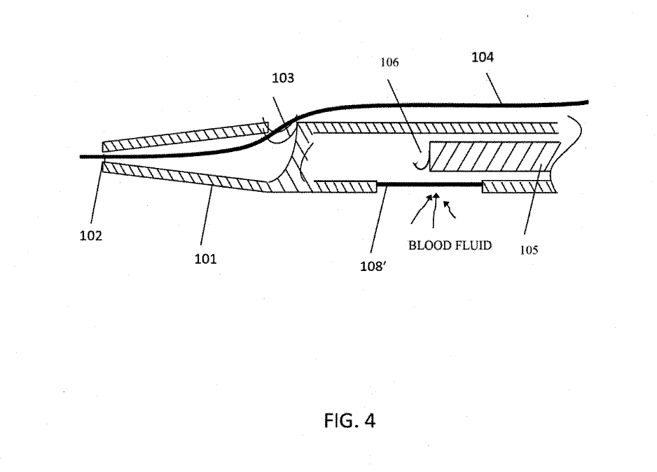

FIG. 4 illustrates a side cross-sectional view of the exemplary measurement catheter according to another exemplary embodiment of the present disclosure. In this exemplary embodiment, the sheath has no openings for the pressure in the vessel to be transmitted through. For example, a region which can be a membrane 108′ that can be a portion of the sheath or if the sheath is compliant, such that the pressure change can cause the deformation of the compliant portion of the sheath or membrane 108′. The compliant portion of the sheath can be or include an inclusion in the sheath, a hole in the sheath filled with a compliant material, or a different sheath material fused to the sheath. The compliant portion of the sheath can be transparent. The first surface and/or the second surfaces of the sheath can be measured by the OCT modality to determine a motion of the sheath, which corresponds to pressure inside the vessel. The redirected beam after the polished of the ball lens can be used for both optical imaging modality and pressure measurement modality. The pressure measurement and OCT imaging can simultaneously or serially. In yet another embodiment there are multiple OCT fibers and multiple pressure measurement configurations that additionally incorporate or are associated with compliant materials within the sheath for transmitting pressure from outside the sheath to inside the sheath.

In yet another exemplary embodiment of the present disclosure, these different compliant portions of the sheath can be configured so that they measure a difference or ratio of pressure across a vascular lesion. According to a further exemplary embodiment of the present disclosure, the electromagnetic radiation provided from one surface of the compliant portion of the sheath can be combined with another electromagnetic radiation from a reference and detected. Such exemplary interference can be analyzed (e.g., using a specifically-programmed computer) with respect to the amplitude or the phase of the resultant radiation to determine, e.g., a displacement of the compliant portion of the sheath which can then be related to a pressure based on at least one of a knowledge of the mechanical properties of the complaint portion of the sheath or a predetermined calibration function.

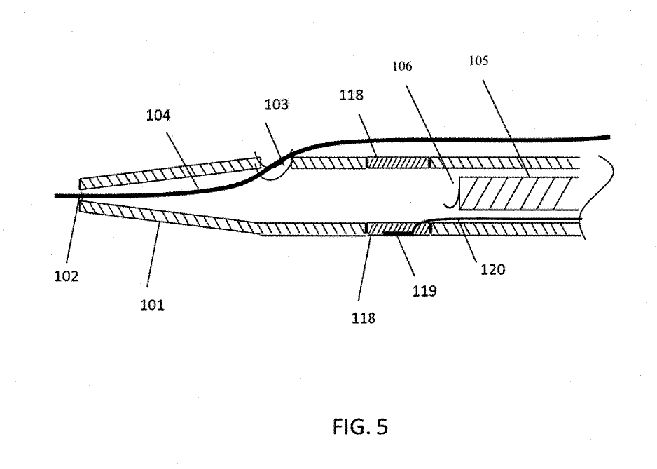

FIG. 5 shows a side cross-sectional view of the exemplary measurement catheter according to still another exemplary embodiment of the present disclosure. In this exemplary embodiment, the pressure sensor can be integrated into the sheath of the catheter. The sheath can be open (as shown in FIG. 5) or sealed. A rigid sheath portion 118 can provide a non-bendable support for a pressure sensor or chip 119, which can be connected to the pressure-reading console by a wire 120. The pressure measurement and/or the OCT imaging can occur and/or be performed sequentially or in parallel over time. The pressure measurement arrangement can include, but does not have to be limited to, e.g., a Fabry Perot interferometer, a fiber Bragg grating, a Rayleigh scattering sensor, a photonic crystal fiber, a birefringent and/or a polarization maintaining fiber or the like.

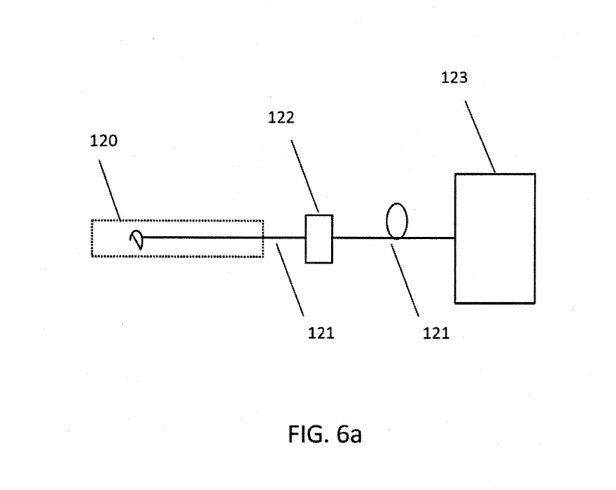

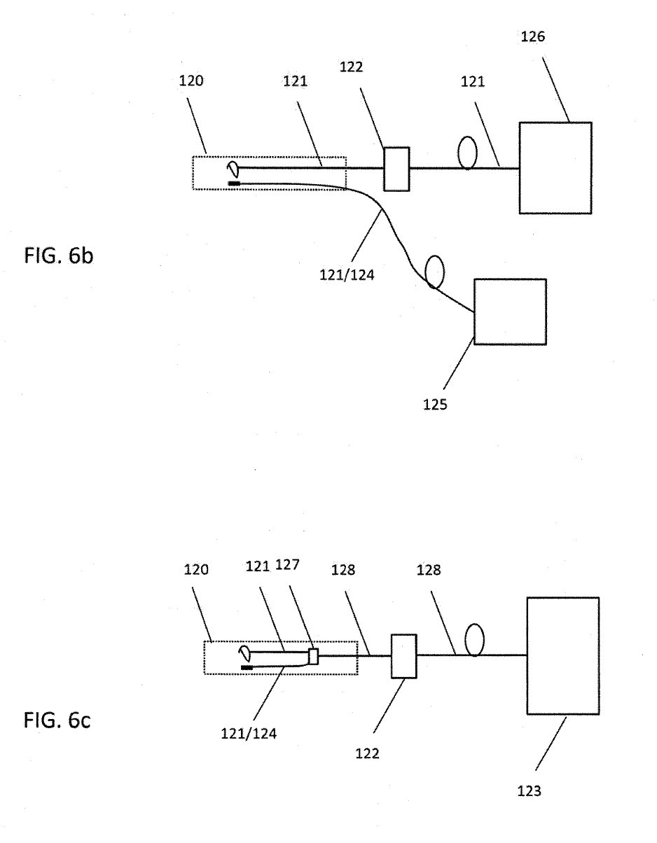

FIGS. 6a-6c illustrate exemplary configuration how the catheter can be connected to system consoles. For example, FIG. 6a shows one exemplary configuration providing the connection between the catheter and the system console for the exemplary embodiments shown in FIGS. 2a, 4 and 5. A single mode fiber 121 exiting from an OCT-FFR catheter 120 can be provided through a rotary junction 122 for a purpose of, e.g., spinning. In addition, a volumetric video can be recorded in an exemplary OCT/FFR console 123.

Further, other exemplary configuration according to certain exemplary embodiments of the present disclosure shown in FIGS. 3a-3d can use one fiber for OCT imaging and another fiber for FFR measurement. Various exemplary methods for the connection between OCT-FFR catheter 120 and the system consoles 123, 125, 126 are illustrated in FIGS. 6b and 6c. For example, as shown in FIG. 6b, the OCT imaging single mode fiber 121 can be connected to the rotary junction 122 for spinning, and then connected to the OCT console 126. The Fiber optic sensor can be connected to FFR console 125. As illustrated in FIG. 6c, the OCT imaging signal and pressure signal can be coupled into a double clad fiber 128 by a combiner 127. The fiber optic sensor is connected to the combiner 127 by either a single mode fiber 121 or a multimode fiber 124. After the combiner 127, the OCT signal can be transmitted through the center core, and the pressure signal can be transmitted through the outer core. After the rotary junction 122, both of the can be sent to the OCT/FFR console 123.

In yet other exemplary embodiments of the present disclosure, additional optical diagnosis or imaging modalities that utilize optical fibers such as fluorescence, time-resolved fluorescence, fluorescence lifetime, absorption spectroscopy, and Raman spectroscopy, etc. can be associated with the pressure sensing arrangement in the housing of the exemplary catheter for combined optical diagnostic capabilities and pressure sensing. These exemplary optical technologies can utilize the same fiber that is used for pressure sensing or via multiple different fibers disposed within the outer housing. Bragg gratings, Raman scattering, photonic crystal fibers or the like can be used in the fiber that can be used to transceive the electromagnetic radiation for exciting fluorescence, inelastic scattering, or detecting absorption within the sample. Such different exemplary modalities can be separated from one another by unique characteristics of the imaging modality radiation with respect to the pressure sensing radiation, such as wavelength or polarization state. When these other imaging modalities utilize multiple wave guiding regions for excitation and detection of radiation from the sample, according to various exemplary embodiments of the present disclosure, the pressure sensing fiber can be provided in one or more of the wave guiding regions. When the electromagnetic radiation with the same properties is used for imaging and pressure sensing, as may be the case of broadband illumination of the sample for spectroscopy, the spectrum returned from a Bragg grating can provide information regarding the pressure. For example, a portion of this spectrum can be utilized for pressure measurement, and a portion can be used for determining the absorption or scattering attenuation provided by the sample. These electromagnetic radiations can be discriminated by path-length determining means such as time-resolved detection or interferometry.

The foregoing merely illustrates the principles of the disclosure. Various modifications and alterations to the described embodiments can be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems including second or higher order harmonic microscopy, sum/difference frequency fluorescence microscopy (one-photon or multi-photon fluorescence), and Raman microscopy (CARS, SRS), and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art can be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

1. An apparatus for obtaining information regarding at least one sample, comprising:
at least one optical data receiver first arrangement which is configured to obtain data for the at least one sample based on a first light radiation provided from the at least one sample;
at least one pressure sensor second arrangement which is configured to measure a pressure of at least one fluid that is provided at or near the at least one sample based on a second light radiation; and
a housing third arrangement at least partially enclosing the first and second arrangements.

2. The apparatus according to claim 1, wherein the at least one second arrangement includes a deformable arrangement.

3. The apparatus according to claim 1, wherein the second light radiation is the same as the second light radiation.

4. The apparatus according to claim 1, wherein the second light radiation is different from the second light radiation.

5. The apparatus according to claim 1, wherein at least one portion of the second light radiation is transmitted through the first arrangement.

6. The apparatus according to claim 1, wherein the third arrangement includes at least one of (i) a deformable arrangement or an aperture, or (ii) at least one channel that is structured to house a guide wire.

7. The apparatus according to claim 1, wherein the second arrangement includes at least one of (i) at least two portions, wherein a first portion of the portions is movable with respect to a second portion of the portions, (ii) a plurality of second arrangement position longitudinally along an extension of the third arrangement, or (iii) a fiber Bragg grating, a Rayleigh scattering fiber, or a photonic crystal fiber.

8. The apparatus according to claim 7, further comprising a detector arrangement which is configured to receive a third light radiation reflected from the first portion and a fourth light radiation reflected from the second portion, wherein the detector arrangement determines a position of the second portion with respect to the first portion based in an interference between the third and fourth light radiations.

9. The apparatus according to claim 8, wherein the position is related to the pressure.

10. (canceled)

11. The apparatus according to claim 1, wherein a diameter of the third arrangement at a portion that at least partially encloses the first and second arrangements is less than 2.6 French.

12. (canceled)

13. The apparatus according to claim 1, wherein the first arrangement includes an interferometer.

14. The apparatus according to claim 13, wherein the interferometer is a Fabry-Perot interferometer.

15. The apparatus according to claim 1, wherein the first arrangement is further configured to perform a spectroscopy.

16. The apparatus according to claim 1, wherein at least one of the first arrangement or the second arrangement are rotatable within the third arrangement.

17. (canceled)

18. The apparatus according to claim 1, wherein the first arrangement is further configured to perform an optical coherence tomography (OCT) procedure.

19. The apparatus according to claim 1, wherein the OCT procedure is at least one of a time domain OCT, spectral-domain OCT or swept-source OCT.

20. The apparatus according to claim 1, wherein the first arrangement obtains data and the second arrangement measures pressure substantially simultaneously.

21. The apparatus according to claim 1, wherein the third arrangement is sized to be insertable into a blood vessel.

22. (canceled)

23. A method for obtaining information regarding at least one sample, comprising:
obtaining data for the at least one sample based on a first light radiation provided from the at least one sample using a first arrangement; and
measuring a pressure of at least one fluid that is provided at or near the at least one sample based on a second light radiation using a second arrangement, wherein the first and second arrangements are at least partially enclosed by a housing arrangement.

24. An apparatus for obtaining information regarding at least one sample, comprising:
at least one optical data receiver which is configured to obtain data for the at least one sample based on a first light radiation provided from the at least one sample;
at least one pressure sensor which is configured to measure a pressure of at least one fluid that is provided at or near the at least one sample using a deformable element based on a second light radiation; and
a housing at least partially enclosing the at least one optical data receiver, wherein the deformable element is provided within or on a surface of the housing.

25. A method for obtaining information regarding at least one sample, comprising:
obtaining data for the at least one sample based on a first light radiation provided from the at least one sample using an optical arrangement; and
measuring a pressure of at least one fluid that is provided at or near the at least one sample based on a second light radiation using a deformable element, wherein the first and second arrangements are at least partially enclosed by a housing, and wherein the deformable element is provided within or on a surface of the housing.
